(19) United States
(12) Patent Application Publication
(10) Pub. No.: US 2010/0272793 A1
(43) Pub. Date: Oct. 28, 2010

(54) Controlled Release Hydraliazine Formulations

(76) Inventor: Navaneeta K. Gorrepati, Desoto, TX (US)

Correspondence Address: Myers Wilson P.C., 16610 Dallas Parkway, Suite 2000, Dallas, TX, 75248, US

(21) Appl. No.: 12/427,863

(22) Filed: Apr. 22, 2009

(51) Int. Cl. A61K 9/64; A61K 31/502; A61K 9/24

(52) U.S. Cl. 424/456; 514/248; 424/473

(57) ABSTRACT

The invention is for a method and composition for preparing a controlled release pharmaceutical formulation which can be used to administer Hydralazine hydrochloride over a 24 hours time frame, and for controlled release oral dosage forms of Hydralazine hydrochloride in the form of a tablet and a capsule.

### CROSS-REFERENCE TO RELATED APPLICATIONS

None.

### TECHNICAL FIELD

The present invention relates to controlled release formulations of Hydralazine which are utilized in the treatment of hypertension in humans.

### BACKGROUND

This invention relates to two controlled release formulations of Hydralazine which are utilized in the treatment of Hypertension.

Hydralazine exerts an antihypertensive effect directly on vascular smooth muscle, producing relaxation of muscle fiber resulting in a decrease in blood pressure. Hydralazine is currently commercially available in the United States for oral use as immediate release tablet which must be administered 3 to 4 times daily.

Controlled release formulations are designed to release significant quantity of drug only at specific timed intervals. The advantages of controlled release products are well known in the pharmaceutical field and include the ability to maintain a desirable blood level over a longer period of time while increasing patient compliance by reducing the number of dosage administrations necessary to achieve the same.

Oral controlled release dosage forms are available as tablets and capsules. To date, no controlled release dosage form of Hydralazine is available in the US market. Both the controlled release tablet and capsule dosage formulations are described in this invention in view of the preference desired by the patient.

### SUMMARY

It is accordingly an object of the invention to provide a method and composition for preparing a controlled release pharmaceutical formulation which can be used to administer Hydralazine hydrochloride over a 24 hours time frame.

It is another object of the invention to provide controlled release oral dosage forms of Hydralazine hydrochloride in the form of a tablet and a capsule.

Pharmaceutically acceptable salts of Hydralazine for use according to the present invention include pharmaceutically acceptable acid addition salts. The hydrochloride salt is particularly preferred.

Formulations of the dosage forms according to the invention utilize Hydralazine or its pharmaceutically acceptable salts in controlled release form. Controlled release systems that are known may be according to the invention include erosion, diffusion or osmosis controlled delivery systems. The drug dissolution may be through a rate controlled matrix or barrier system. Controlled release matrices containing diffusion controlled polymers with suitable hydrophilic and hydrophobic adjuvant are added. In osmotic controlled systems release of the drug is controlled by the permeability of the membrane and the osmotic pressure generated by core matrix. The release of the drug may also be p^{H} or time controlled.

A suitable matrix comprises one or more of polymethacrylates such as Eudragit® FS 30D, Eudragit® L 30D-55 and Eudragit® NM 30D. Preferably the matrix contain between 10% and 50% by weight of the formulation. Additionally the matrix may also contain suitable quantities of other materials, e.g., diluents, binders, glidants, lubricants, colorations, that are conventional in pharmaceutical art.

The Hydralazine containing controlled release matrix of the invention can be prepared by distributing the ingredient in the controlled release system using conventional pharmaceutical techniques such as wet granulation, dry granulation or dry mixing.

In another embodiment of the present invention, the Hydralazine is incorporated in the form of controlled release beads. Typically the Hydralazine is bound to an inert carrier such as starch or sugar spheres. The spheres may be prepared of any pharmaceutically acceptable inert ingredients. A binding agent such as water soluble cellulose (for example hydroxylpropylmethylcellulose) or polyvinylpyrrolidone is utilized to apply Hydralazine to the inert spheres along with suitable acidifying agents such as fumaric acid, citric acid, malic acid, adipic acid etc., and other glidants such as talc etc. The Hydralazine coated beads are further coated with polymeric coating which permits release of Hydralazine at a controlled rate in an aqueous medium. Suitable coating materials include insoluble celluloses, particularly ethyl cellulose for example Surelease® and polymethacrylates for example Eudragit polymers™. Additionally the coating may also contain suitable plasticizer, antifoaming agent, anti-adherent and colorants etc.

The amount of controlled release coating material is dependent on the desired release rate but is generally in the range of 5% to 25% weight of the Hydralazine coated beads.

For once daily administration the dosage forms conveniently contain 25 mg to 200 mg of Hydralazine or a pharmaceutically acceptable salt preferably Hydralazine hydrochloride. Compositions according to the invention may be compressed into tablets using conventional pharmaceutical techniques or filled into capsules.

The following examples disclose various aspects of the present invention. They are not to be construed to limit the claims in any manner whatsoever.

### EXAMPLE 1

A controlled release tablet formulation capable of slowly releasing Hydralazine hydrochloride having the following composition is prepared as described below.

| | % w/w |
|---|---|
| Hydralazine HCI | 29.2 |
| Dicalcium phosphate, dihydrate | 49.9 |
| Eudragit NM 30D | 20.1 |
| Colloidal silicon dioxide | 0.3 |
| Magnesium stearate | 0.5 |

Hydralazine HCI and Dicalcium phosphate are mixed and granulated with Eudragit NM 30D in a fluid bed granulator and subsequently dried. The dried granulation is passed through a 20 mesh screen, and further mixed with colloidal silicon dioxide and magnesium stearate in a blender. The blend is compressed into tablets of different strengths of Hydralazine having the following composition.

| | Hydralazine | Hydralazine | Hydralazine | Hydralazine |
|---|---|---|---|---|
| | 200 mg | 100 mg | 50 mg | 25 mg |
| | mg/tablet | mg/tablet | mg/tablet | mg/tablet |
| Hydralazine | 245.6 | 122.8 | 61.4 | 30.7 |
| HCI | | | | |
| Dicalcium | 418.8 | 209.4 | 104.7 | 52.34 |
| Phosphate, | | | | |
| dihydrate | | | | |
| Eudragit NM | 169 | 84.5 | 42.25 | 21.13 |
| 30D | | | | |
| Colloidal | 2.4 | 1.2 | 0.6 | 0.3 |
| silicon dioxide | | | | |
| Magnesium | 4.2 | 2.1 | 1.05 | 0.53 |
| stearate | | | | |

### EXAMPLE 2

A controlled release formulation of beads capable of slowly releasing Hydralazine hydrochloride having the following composition is prepared as described below.

| | % w/w |
|---|---|
| Hydralazine HCI | 51.4 |
| Methocel E5 | 1.0 |
| Purified water | QS |
| Sugar spheres | 35.2 |
| Surelease | 11.5 |
| Talc | 0.9 |

Methocel E5 is dissolved in sufficient water and then Hydralazine HCI and Talc are mixed into the solution. Sugar spheres are coated with the above solution in a fluid bed coater and dried. Surelease is applied to the Hydralazine beads in the fluid bead coater and dried. The controlled release beads prepared above are filled into hard gelatin capsules to produce different strengths of Hydralazine having the following composition.

| | Hydralazine | Hydralazine | Hydralazine | Hydralazine |
|---|---|---|---|---|
| | 200 mg | 100 mg | 50 mg | 25 mg |
| | mg/capsule | mg/capsule | mg/capsule | mg/capsule |
| Controlled | 480 | 240 | 120 | 60 |
| Release Beads | | | | |
| of Hydralazine | | | | |
| Hard gelatin | Size 0 | Size 1 | Size 2 | Size 4 |
| Capsules | | | | |

It should be understood that many modifications and variations can be made in the proportions and components used herein without departing from the spirit scope of the invention which is solely defined by the claims.

### BRIEF DESCRIPTION OF THE DRAWINGS

None.

What is claimed is:

1. A controlled release oral tablet dosage form for the treatment of hypertension in humans, comprising 25 mg to: 200 mg of Hydralazine hydrochloride or a pharmaceutically acceptable salt thereof and wherein the composition comprises a disintegrating matrix, a non-disintegrating matrix or an erodible matrix.

2. The pharmaceutical composition according to claim 1, wherein the tablet matrix compromises polymethacrylates, Dicalcium phosphate, colloidal silicon dioxide and magnesium stearate.

3. A controlled release tablet formulation of Hydralazine hydrochloride consisting essentially of:
(a) a core consisting essentially of:
(i) about 5-50% Hiydralazine HCI;
(ii) about 10-60% of a compound selected from the group consistilg of Dicalcium phosphate and calcium sulfate;
(iii) about 5-40% of a compound selected from the group consisting of Eudragit NM 30D , Eudragit FS 30D, and Eudragit L 30D-55;
(iv) about 0.1-5% of a compound selected from the group eonsisting of colloidal silicon dioxide, starch and talc;
(v) about 0.1-5% a compound selected from the group consisting of magnesium stearate, calcium stearate and stearic acid.
(b) optionally, a seal coat around the core;
(c) a semipermeable membrane coating covering said core comprising; and
(d) at least one passageway in the semipermeable membrane to allow release of the Hydralazine hydrochloride from the core to the environment of use to provide therapeutic levels of Hydralazine hydrochloride for from 12 to 24 hours.

4. A controlled release hard gelatin capsule dosage form for the treatment of hypertension in humans, comprising 25 mg to 200 mg of Hydralazine hydrochloride or a pharmaceutically acceptable salt thereof and wherein composition comprises of controlled release beads.

5. The pharmaceutical composition according to claim 4, where the beads in the capsule comprise sugar spheres, hydroxylpropylmethylcellulose, Surelease and talc.

6. A controlled release capsule formulation of Hydralazine hydrochloride, consisting essentially of:
(a) a core consisting essentially of:
(i) about 5-50% of Hydralazine HCI;
(ii) about 25-50% of a compound selected from the group consisting of sugar spheres and microcrystalline cellulose spheres;
(iii) about 0.1-5% of a compound selected from the group consisting of Hydroxylpropylmethylcellulose and polyvinylpyrrolidone;
(iv) about 5-25% of a compound selected from the group consisting of Surelease and Aquacoat;
(v) about 0.1-5% of a compound selected from the group consisting of Talc and colloidal silicon dioxide; and
(vi) about size 0-5 of a compound selected from the group consisting of hard gelatin capsules and Hydroxylpropylmethylcellulose capsules.
(b) optionally a seal coat around the core;
(c) a semipermeable membrane coating coversing said core comprising; and
(d) at least one passageway in the semipermeable membrane to allow release of the Hydralazine hydrochloride from the core to the environment of use to provide therapeutic levels of Hydralazine hydrochloride for from 12 to 24 hours.
